(19) United States
(12) Patent Application Publication
(10) Pub. No.: US 2024/0307346 A1
(43) Pub. Date: Sep. 19, 2024

(54) METHOD FOR TREATING OR PREVENTING A DISEASE OF INCOMPLETE IMMUNE RECONSTITUTION IN AIDS USING (5R)-5-HYDROXYTRIPTOLIDE

(71) Applicants: PEKING UNION MEDICAL COLLEGE HOSPITAL, Beijing (CN); SHANGHAI PHARMACEUTICALS HOLDING CO., LTD., Shanghai (CN)

(72) Inventors: Taisheng LI, Shanghai (CN); Yanjun LIU, Shanghai (CN)

(21) Appl. No.: 18/606,090

(22) Filed: Mar. 15, 2024

Related U.S. Application Data
(63) Continuation-in-part of application No. 16/969,594, filed on Aug. 13, 2020.

(30) Foreign Application Priority Data
Feb. 13, 2018 (CN) 201810149149.4

(51) Int. Cl. A61K 31/365; A61P 31/18; A61P 37/06

(52) U.S. Cl. CPC A61K 31/365; A61P 31/18; A61P 37/06

(57) ABSTRACT

Disclosed in the present invention is a method for treating or preventing a disease of incomplete immune reconstitution in aids using (5R)-5-hydroxytriptolide, in particular a method for treating or preventing a disease of incomplete immune reconstitution in AIDS in a subject in need thereof, comprising administering a therapeutically effective amount of a medicament comprising (5R)-5-hydroxytriptolide to the subject. The present invention uses (5R)-5-hydroxytriptolide (T8) in experiments and finds that (5R)-5-hydroxytriptolide has an immunosuppressive activity against incomplete immune reconstitution in AIDS, is characterized by high efficiency and low toxicity, and has a good safety treatment index.

### CROSS-REFERENCE TO RELATED APPLICATIONS

The application is a Continuation-in-part Application of U.S. application Ser. No. 16/969,594, filed on Aug. 13, 2020, filed as International Application No. PCT/CN2018/121201, filed on Dec. 14, 2018, which claims priority of Chinese Patent Application CN201810149149.4 filed on Feb. 13, 2018, the contents of which are incorporated herein by reference in their entireties.

### TECHNICAL FIELD

The present disclosure a method for treating or preventing a disease of incomplete immune reconstitution in aids using (5R)-5-hydroxytriptolide

### BACKGROUND

Tripterygium wilfordii Hook f (Lei Gong Teng) is a kind of vine plant of Celastraceae family, and mainly grows under humid environment near the mountain forest in the Yangtze River basin and southwest area of China. Its major chemical components include diterpenes, triterpenes, sesquiterpenes, alkaloids and the like. The pharmacological study in the last twenty years demonstrated that the extract of Tripterygium wilfordii Hook f has anti-inflammatory, immunosuppressive, antifertility, anti-tumor and antibacterial activities. Especially in respect of immunosuppressive function, triptolide in Tripterygium wilfordii Hook f was found to have significant biological activity. However, the clinical uses of triptolide are limited by its toxicity and side effects. The inventors of patent CN1223595C disclosed a series of novel triptolide derivatives through chemical modification on structure of this compound after studying the structure-activity relationship of triptolide thoroughly, which can be used as immunosuppressant for prevention and therapy against autoimmune diseases, infectious diseases and the like. Especially, it was demonstrated that (5R)-5-hydroxyltriptolide (LLDT-8, referred to as T8, having a structure as shown below), had anti-inflammatory and immunosuppressive activity with high potency but low toxicity and a good safety index, when evaluated in many in vitro assays and in vivo animal models.

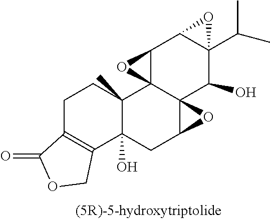 [SMILES: CC(C)[C@]12O[C@H]1[C@@H]1O[C@]13[C@]1(O[C@H]1C[C@@]1(O)C4=C(CC[C@]31C)C(=O)OC4)[C@@H]2O]

AIDS (Acquired Immune Deficiency Syndrome) is an infectious disease caused by human immunodeficiency virus (HIV) infection, which is characterized by HIV specifically attacking helper T lymphocytes (CD4⁺), causing the progressive malfunction of human immune system, and eventually causing various opportunistic infections and corresponding tumors. The highly active antiretroviral therapy (HAART) used since the 1990s can significantly reduce the plasma HIV load, and can effectively increase the CD4⁺T-cells count, enabling patients to develop immune reconstitution (For AIDS patients, immune reconstitution refers to the process of recovery of immune system function), thereby reducing the occurrence of AIDS-related diseases and fatality rate.

However, not all HIV-infected people can achieve good immune reconstruction after HAART treatment. Studies have shown that about 15%˜30% of AIDS patients, especially those with advanced HIV infection who have been infected for a long time at the beginning of treatment, cannot achieve ideal immune reconstruction after ART, even if the virus is completely suppressed for a long time. This phenomenon is called a disease of incomplete immune reconstruction in AIDS. The disease of incomplete immune reconstruction in AIDS is a specific disease that occurs in AIDS patients, which is totally different from the disease of AIDS. Furthermore the treatment method of incomplete immune reconstruction in AIDS is also completely different from the treatment of AIDS.

The patients with a disease of incomplete immune reconstitution in AIDS are also called INRs (immunological non-responders). For AIDS patients, INRs refer to patients who had received effective ART for at least 3 years and had plasma viral load below level of detection for at least 2 years, with their CD4 counts consistently below 350 cells/mm³.

Abnormal immune activation refers to a long-term abnormally increased activation state of the immune system. Abnormal immune activation and inflammation caused by HIV infection is an important pathogenesis of non-AIDS-related diseases and incomplete immune reconstitution. Inhibiting excessive immune activation and inflammation may promote the recovery of CD4⁺T lymphocytes in AIDS patients and reduce the occurrence of non-AIDS-related diseases.

However, excessive inhibition may lead to aggravation of incomplete immune reconstitution, so it is important to find a drug that can inhibit abnormal immune activation without aggravating incomplete immune reconstitution. In terms of a drug, in addition to the therapeutic effect, the medication safety thereof also needs to be considered. At present, there are no effective drugs in China and abroad for the treatment or prevention of a disease of incomplete immune reconstitution occurring in AIDS patients (which is hereinafter referred to as incomplete immune reconstitution in AIDS).

### CONTENT OF THE PRESENT INVENTION

The immune activation caused by HIV is different from the body's immune response and immune activation after viral infection in the common sense. Current research shows that its main mechanisms include:
1. Direct pathway: HIV gene products (such as gp120) directly activate lymphocytes and macrophages:
2. Indirect pathway: During HIV infection, because of the state of body's immune deficiency caused by HIV infection, some persistently infected pathogens in the body such as cytomegalovirus (CMV) and Epstein-Barr virus will undergo reactivation and replication, thereby causing immune activation:
3. Translocation of intestinal flora: The loss of a large number of CD4⁺T-cells in the mucosal lymphatic tissue will cause the destruction of the immune components in the intestinal mucosal barrier, which will lead to the translocation of intestinal flora, thereby causing immune activation.
4. Decrease of regulatory T-cells (Treg): Treg plays an important role in maintaining the homeostasis of the body's immune system, and serve a role in down-regulating the immune activation state. Its activation and transformation are closely related to immune activation and immune reconstitution. Studies have shown that CD4⁺CD25⁺Treg in the HIV-infected long-term non-progressors is significantly higher than typical progressors, and the proportion of Treg is negatively correlated with the activated CD8⁺T-cell subsets.
5. CD4⁺T-cells pyroptosis: A study published by Gilad Doitsh et al. in Nature in 2014 showed that the vast majority (95%) of CD4⁺T-cells death caused by HIV was accompanied by the release of inflammatory factors and intracellular contents such as IL-1β, and further induced caspase-1 mediated cell pyroptosis and promoted the accumulation, infection and pyroptosis of uninfected CD4⁺T-cells in the body, thereby forming a vicious circle “pyroptosis—inducing the aggregation of uninfected CD4⁺T-cells—infecting new CD4⁺T-cells-new round of pyroptosis”. In 2015, in a study published on Cell Reports by Galloway et al., a series of experiments proved that the direct contact (formation of synaptic connections) between infected CD4⁺T-cells and uninfected CD4⁺T-cells was a necessary condition to cause virus transmission and cell pyroptosis, and cell-free HIV virions, even with a high load, could not directly cause cell pyroptosis.

Persistent immune activation can have a series of serious consequences, including persistent HIV replication and immune reconstitution disorders, especially the increase in the mortality rate of AIDS patients, which cannot be completely eliminated even if the patients are subjected to a long-term effective antiviral therapy. The consequences mainly include the following several aspects:
1. Virus replication: The direct result of T-cell activation is the production of intracellular NF-κB, which increases the transcription of viral genes integrated into the host gene and produces new virus particles, thereby falling into a vicious circle.
2. Apoptosis: For activated T-cells, immune activation means that they will undergo differentiation, transformation and even apoptosis. Apoptosis related to immune activation is also considered to be an important reason for the decrease of peripheral CD4⁺T-cells.
3. Immune senescence and exhaustion: In the adaptive immune response, naive T-cells are activated after contacting with antigen presenting cells, thereby proliferate and differentiate into effector T-cells and memory T-cells. However, persistent immune activation caused by persistent antigen stimulation or other mechanisms can cause effector T-cells to lose their function. This phenomenon is called “immune exhaustion”. In addition, the thymus of the infected person also shrank to a degree disproportionate to their age, which means that the T-cells developed in the thymus cannot replenish the T-cells consumed in the periphery in time. Immune activation can also lead to the decline of bone marrow hematopoietic function. lymphatic tissue fibrosis, and the like.
4. Immune reconstitution obstacles: In immune non-responders, although antiviral therapy is effective and plasma virus replication is basically inhibited, there are still residual viruses, which may come from virus reservoirs (mainly memory T-cells) and sustainably low levels of virus replication. These residual viruses and their low-level replication are related to persistent immune activation and incomplete immune reconstitution.
5. Other abnormalities related to inflammation such as osteoporosis, atherosclerosis, neurocognitive degeneration, aging, and the like, are now also considered to be related to immune activation, leading to a significant increase in patient mortality.

Abnormal immune activation and inflammation caused by HIV infection is an important pathogenesis of non-AIDS-related diseases and incomplete immune reconstitution. Inhibiting excessive immune activation and inflammation may promote the recovery of CD4⁺T lymphocytes in AIDS patients and reduce the occurrence of non-AIDS-related diseases. However, excessive inhibition may lead to aggravation of incomplete immune reconstitution, so it is important to find a drug that can inhibit abnormal immune activation without aggravating incomplete immune reconstitution. In terms of a drug, in addition to the therapeutic effect, the medication safety thereof also needs to be considered. The present inventors have inventively studied and found that (5R)-5-hydroxytriptolide (T8) inhibits excessive immune activation and inflammation in AIDS patients, and discovered a novel medicament for the treatment of abnormal immune activation or incomplete immune reconstruction in AIDS patients and for the treatment of non-AIDS related diseases.

In addition, the activation of the NF-κB pathway is essential for HIV replication and potential reactivation, and the NF-κB pathway controls the expression of multiple inflammatory factors in the cells.

The present disclosure discovers that (5R)-5-hydroxytriptolide (T8) can inhibit cellular immune activation by inhibiting NF-κB activity, and (5R)-5-hydroxy triptolide (T8) inhibits CD4⁺T-cells subset activation and proliferation.

The present disclosure provides a method for treating or preventing a disease of incomplete immune reconstitution in AIDS in a subject in need thereof, comprising administering a therapeutically effective amount of a medicament comprising (5R)-5-hydroxytriptolide to the subject.

In the present disclosure, the incomplete immune reconstitution is associated with abnormal immune activation (LIU Yu-chao; LI Tai-sheng. Immune Activation and Incomplete Immune Reconstitution in Chronic Human Immunodeficiency Virus Infected Patients, Medical Journal of Peking Union Medical College Hospita, Vol. 8, No. 4-5, p100-104, 2017).

In the present disclosure, the abnormal immune activation is preferably NF-κB-related abnormal immune activation.

In the present disclosure, the abnormal immune activation is preferably chronic abnormal immune activation.

In the present disclosure, wherein the subject is an AIDS patient who has received effective ART for at least 3 years and has plasma viral load below level of detection for at least 2 years, with their CD4 counts consistently below 350 cells/mm³, the CD4 counts are measured using CytoFlex flow cytometer in vitro before the administration.

Preferably, the subject is an AIDS patient who has received effective ART for at least four years and has plasma viral load below level of detection for at least 3.5 years, with their CD4 counts consistently below 350 cells/mm³.

In the present disclosure, wherein the method is to treat or prevent the disease of incomplete immune reconstitution in AIDS by enhancing CD4 counts, the CD4 counts are measured using CytoFlex flow cytometer in vitro before and after the administration.

Preferably, the change of CD4 counts is 49 cells/mm³.

In the present disclosure, the form of (5R)-5-hydroxytriptolide is not particularly limited, which can be solid tablet, liquid, gel, semi-liquid or aerosol form. Solid tablet is preferably.

In the present disclosure, the (5R)-5-hydroxytriptolide is preferably one of the active ingredients or the only active ingredient.

In the present disclosure, the (5R)-5-hydroxytriptolide is administered orally.

In the present disclosure, the (5R)-5-hydroxytriptolide is administered as 0.5 mg-1 mg daily.

In the present disclosure, the (5R)-5-hydroxytriptolide is administered as 0.5 mg or 1 mg daily.

The present disclosure also provides a method for enhancing CD4 counts in a subject in need thereof, comprising administering a therapeutically effective amount of a medicament comprising (5R)-5-hydroxytriptolide to the subject.

In the present disclosure, the subject is the patient with a disease of incomplete immune reconstitution in AIDS.

In the present disclosure, the subject is an AIDS patient who has received effective ART for at least 3 years and has plasma viral load below level of detection for at least 2 years, with their CD4 counts consistently below 350 cells/mm³, the CD4 counts are measured using CytoFlex flow cytometer in vitro before the administration.

Preferably, the subject is people living with HIV, who has received effective ART for at least four years and has plasma viral load below level of detection for at least 3.5 years, with their CD4 counts consistently below 350 cells/mm³.

In the present disclosure, the above preferred conditions can be combined arbitrarily to obtain preferred embodiments of the present disclosure.

The reagents and raw materials used in the present disclosure are all commercially available.

The positive and progressive effects of the present disclosure are: The present disclosure discovers that (5R)-5-hydroxytriptolide (T8) has immunosuppressive activity with high potency but low cytotoxicity in abnormal immune activation in AIDS or incomplete immune reconstitution associated with abnormal immune activation in AIDS, and has a good safety index after assays with (5R)-5-hydroxytriptolide (T8). And T8 enhanced CD4 recovery and alleviated inflammation in long-term suppressed INRs, providing a potential therapeutic option for HIV INR patients. The benefit was superior with LLDT-8 dosage 1 mg daily, and in older participants. Future larger size of clinical studis and pharmacological research into underlying mechanisms are needed to better understand its effectiveness. T8 can inhibit abnormal immune activation without aggravating incomplete immune reconstitution.

### BRIEF DESCRIPTION OF THE DRAWINGS

FIG. 1 shows the inhibition results of NF-κB activity by (5R)-5-hydroxytriptolide.

FIG. 2 shows the inhibition results of CD4⁺T-cells proliferation by (5R)-5-hydroxytriptolide.

FIG. 3 shows the inhibitory effect of (5R)-5-hydroxytriptolide on the activation of CD4⁺T-cell subsets.

FIG. 4 shows the cytotoxicity test of (5R)-5-hydroxytriptolide (T8) and the reference substance (triptolide).

FIG. 5 shows the inhibitory effect test results of (5R)-5-hydroxy triptolide (T8) and the reference substance (triptolide) on the activity of NF-κB signaling pathway.

FIGS. 6A and 6B show the inhibitory effect test results of (5R)-5-hydroxytriptolide (T8) and the reference substance (triptolide) on the immune activation of CD4⁺T-cell subsets. CD4⁺T-cells were isolated from the freshly isolated healthy human peripheral blood cell PBMCs using anti-CD4 antibody-coated magnetic beads through positive screening. The cells were stimulated with PMA (100 nM) and Ionomycin (1 μM) or anti-CD4 antibody-coated magnetic beads for 72 hours, and T8 or triptolide (100 ng/mL) was added at the same time. The cells were collected, stained with anti-CD4-PE antibody to gate CD4⁺T-cells, and the expression of CD38 and HLA-DR on the cell surface was detected by flow cytometry. FIG. 6A shows the results of flow cytometry in multiple completely independent repeated tests: FIG. 6B shows the statistical results of flow cytometry, counting the cell-positive rate and calculating the mean fluorescence intensity (MFI), respectively. Paired student t-test was used to analyze the significant differences.

FIG. 7 shows the inhibitory effects of T8 or triptolide on the proliferation of CD4⁺T-cells. PBMCs were stained with CFSE: PHA-P (5 μg/mL) was added to induce the cell proliferation, and the test compound T8 or triptolide (100 ng/ml) was added at the same time: after 72 hours, the cells were collected and stained with anti-CD4-PE antibody. Flow cytometry was used to detect CFSE and analyze cell proliferation. (A) Test results of flow cytometry in three completely independent repeated tests: (B) Statistical results of flow cytometry. Paired student t-test was used to analyze the significant differences.

### DETAILED DESCRIPTION OF THE EMBODIMENT

The following examples further illustrate the present disclosure, but the present disclosure is not limited thereto. In the following examples, the experimental methods without specific conditions are carried out according to conventional methods and conditions, or according to the product specification.

### Example 1: The Inhibitory Effects of T8 on the Activity of NF-κB

NF-κB driven luciferin expression plasmid (p3κB-luc, 100 ng) and internal reference plasmid pBgal (purchased from Bioonline: www.bioon.com.cn) (pSV-β-Galactosidase Control Vector, β-galactosidase expression plasmid, 5 ng) were transfected into HEK293T cells (purchased from ATCC). After 24 hours, the cells were treated with TNF-alpha (20 ng/mL) or untreated, and with or without the addition of T8 compound (100 nM). After another 24 hours, all cells were collected and lysed with reporter lysis buffer (purchased from Promega). Luciferase assay system (Promega) was used to detect luciferase activity. Beta-Glo Asaay system (Promega) was used to detect β-Galactosidase activity, and the inhibition effects of T8 on NF-κB activity was analyzed.

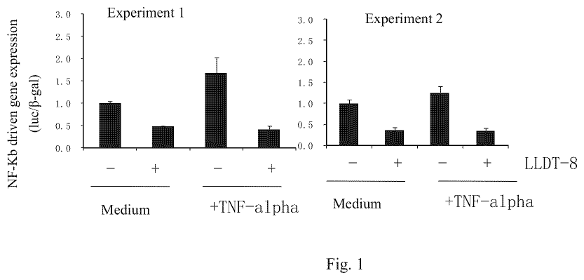

The test results are shown in FIG. 1. The test results show (5R)-5-hydroxytriptolide has inhibitory effect on NF-κB activity. (5R)-5-hydroxytriptolide can not only effectively inhibit the background NF-κB activity (untreated with TNF-alpha), but also effectively inhibit NF-κB activity stimulated by TNF-alpha.

### Example 2: The Inhibitory Effects of T8 on the Proliferation of CD4⁺T-Cells

Healthy human peripheral blood mononuclear cells (PBMC) (purchased from ATCC) were cultured in RPMI-1640/10% FBS serum medium containing IL-2 (20IU). and then stained with CFSE (carboxyfluorescein diacetate succinimidy ester) (10 nM) at 37° C. for 10 minutes, washed twice with the above medium, followed by addition of PHA-P (phytohemagglutinin P) (5 μg/mL) to stimulate cell proliferation with or without addition of T8 compound (100 nM). After 48 hours of culture, the cells were collected, stained with anti-human CD4 flow cytometry antibody (purchased from Invitrogen) (4° C. 30 min), and CD4⁺T-cells were gated by flow cytometry to analyze cell proliferation by detect the CFSE staining intensity.

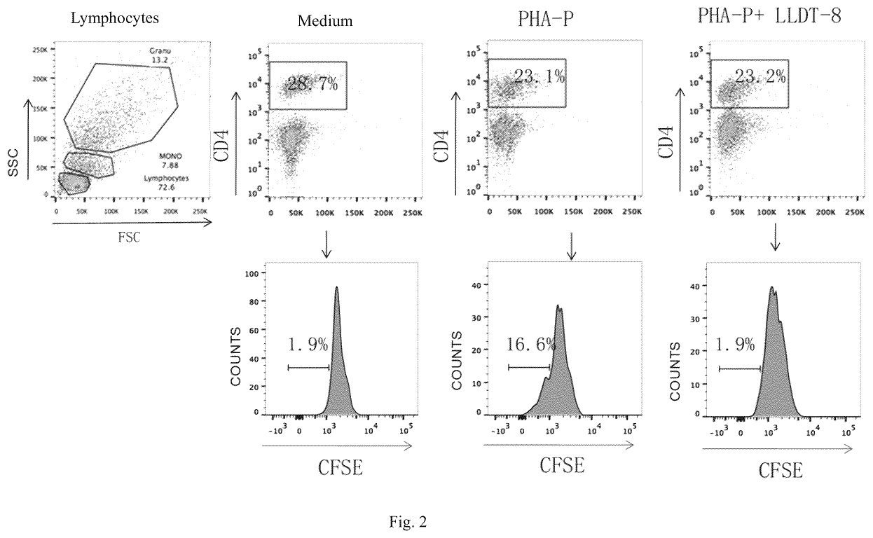

The test results are shown in FIG. 2. The test results show that (5R)-5-hydroxytriptolide inhibits the proliferation of CD4⁺T cells stimulated by PHA (cell proliferation is reduced from 16.6% to background proliferation 1.9%).

### Example 3: The Inhibitory Effect of T8 on the Activation of CD4⁺T-Cell Subsets

Healthy human PBMC (1×107 cells) were stimulated with PMA (propylene glycol monomethyl ether acetate) (20 nM) and Ionomycin (1 μM) at 37° C. with or without the addition of T8 compound (100 nM). After 48 hours, the cells were stained with anti-human CD4, CD38, HLA-DR flow cytometry antibody (purchased from Invitrogen), and the CD4⁺T-cells were gated by flow cytometry to detect the expression of CD38, HLA-DR molecules on the cell surface.

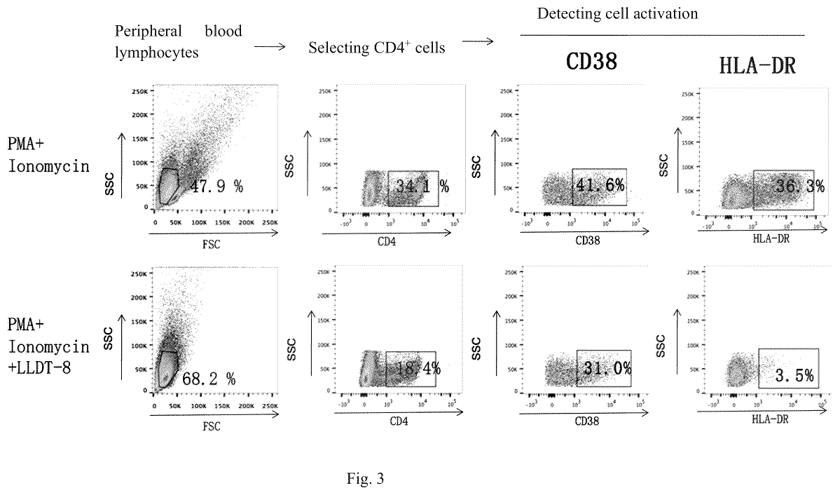

The test results are shown in FIG. 3. The test results show that (5R)-5-hydroxytriptolide inhibits the activation of the cells stimulated by PMA+Ionomycin (CD38CD4/CD4 decreased from 41.6% to 31%; HLA-DRCD4/CD4 decreased from 36.3% to 3.5%).

### Comparative Example 1: The Cytotoxicity Tests of (5R)-5-Hydroxytriptolide (T8) and the Reference Substance (i.e., Triptolide)

### 1. Experimental Materials

### 1) Compounds to be Tested

(5R)-5-hydroxytriptolide (T8) and the reference substance (triptolide), provided by Shanghai Pharmaceuticals Holding Co., LTD.: white powder, purity >99%, stored at 4° C. for later use:

Preparation methods: DMSO was used to prepare mother liquor, and medium (RPMI1640, Gibco) was used to prepare the working solution. The final concentration of DMSO during cell culture was <0.02%, which had no effect on cell growth.

### 2) Cells and Reagents

HEK293T cells were cultured in DMEM complete medium containing 10% fetal bovine serum, 100 U/mL penicillin and 100 μg/mL streptomycin:

DMEM medium and fetal bovine serum were purchased from Gibco; Penicillin and streptomycin were purchased from Invitrogen;

MTT [3-(4,5-dimethylthiahiazol-2-yl)-2,5-diphenyltetrazolium bromide], SDS (Sodium dodecyl sulfate) and DMF (N,N-dimethylformamide) were purchased from Sigma:

MTT (Formazan) solution (100 mL): 10 g of SDS (Sodium dodecyl sulfate), 50 mL of DMF (N,N-dimethylformamide) and 50 mL of H₂O were mixed, heated, stirred evenly, and stored at 4° C.

### 2. Experimental Method

MTT colorimetric method was used to detect the cytotoxicity. HEK293T cells were seeded into 96-well plates (1×10⁴/100 μL/well), 100 μL of each concentration of the compounds (compound concentration refers to the figures) was added in triplicate. Control wells without compound were setting at the same time. The cells were incubated in a 37° C. 5% CO₂ cell incubator for 72 hours. 100 μL of the supernatant was aspirated from each well and discarded, followed by addition of 20 μL of MTT solution (5 mg/mL) into the wells. The cells were incubated in a 37° C., 5% CO₂ cell incubator for 4 hours, followed by addition of 100 μL of Formazan solution to each well and incubation overnight in the incubator until all Formazan was found to be dissolved under a common optical microscope. The absorbance was measured at 595 nm. The CC₅₀ value (50% Cytotoxic Concentration) was calculated, i.e., the concentration of the compound required for producing toxicity on 50% of HEK293T cells.

### 3. Experimental Results

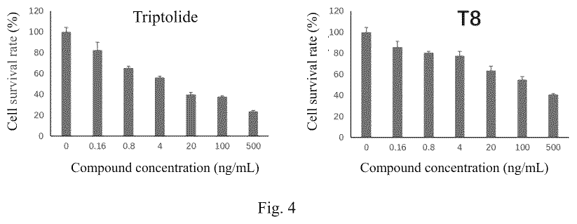

HEK293T cells were used to detect the cytotoxicity of the two compounds. It is found that the CC₅₀ (50% Cytotoxic Concentration) value of (5R)-5-hydroxytriptolide (T8) on HEK293T cells is about 435 ng/ml, while the CC₅₀ value of the reference substance (triptolide) on HEK293T cells is about 17 ng/ml, indicating that T8 has lower cytotoxicity compared with triptolide (see FIG. 4 for details).

### Comparative Example 2: The Inhibitory Effects of (5R)-5-Hydroxytriptolide (T8) and the reference substance (triptolide) on the activity of NF-κB signaling pathway.

### 1. Experimental Materials

### 1) The Compounds to be Tested Were the Same as Above.

### 2) Cells and Reagents

HEK293T cells were cultured in DMEM complete medium containing 10% fetal bovine serum. 100 U/mL penicillin and 100 μg/mL streptomycin.

DMEM medium, fetal bovine serum and OPTI-MEM were purchased from Gibco: Penicillin and streptomycin were purchased from Invitrogen: TNF-α was purchased from R&D: Lipofectamine 2000 Transfection Reagent was purchased from Life Technologies: Luciferase assay system and Glo Asaay system were purchased from Promega.

The pSV-β-Galactosidase Control Vector plasmid was from Promega (CATALOG #E1081).

### 2. Experimental Method

The NF-κB reporter gene plasmid p3κB-luc was transfected into HEK293T cells for expression, and pSV-β-Galactosidase Control Vector was co-transfected as transfection internal reference. The activation of NF-κB was induced by TNF-α treatment. T8 and triptolide were added respectively. The expression of NF-κB reporter gene was indicated by detecting luciferase activity, and the effect of T8 and triptolide on NF-κB activation induced by TNF-α was evaluated.

Cell transfection: HEK293T cells were transfected with lipofectamine 2000 Transfection Reagent according to the reagent instructions. 100 ng of plasmid was transfected with 1 μL of transfection reagent into each well of a 24-well cell cultureplate. The plasmid and the transfection reagent were diluted with 50 pL of OPTI-MEM medium, respectively and then mixed together. The mixture was allowed to stand at room temperature for 5 minutes, and then added into the cells in each well. The resulting mixture was mixed gently, placed back into the 37° C. incubator, and the cells were cultured for a specified time.

Luciferase activity detection: NF-κB reporter gene plasmid p3κB-luc plasmid (100 ng), internal reference plasmid pSV-β-Galactosidase Control Vector (5 ng) were transfected into HEK293T cells. After 24 hours. TNF-α (20 ng/ml) was added (or not added) to stimulate the cells for 24 hours, and then the cells were collected and lysed with reporter lysis buffer. Luciferase assay system was used to detect luciferase activity. and Beta-Glo Asaay system was used to detect β-Galactosidase activity.

### 3. Experimental Results

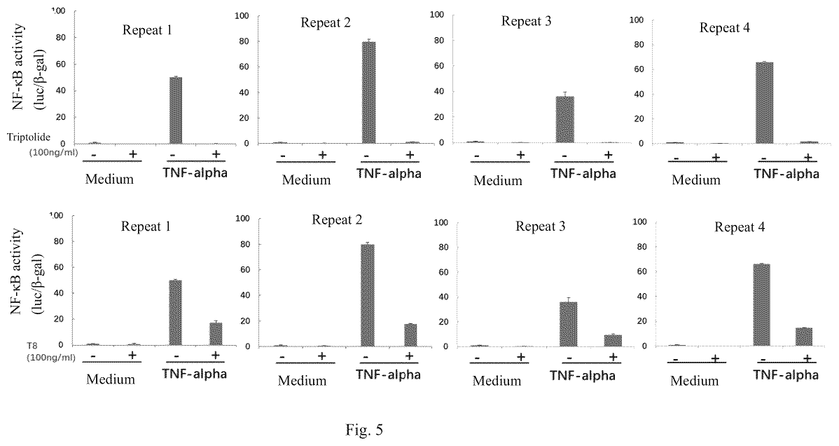

According to FIG. 5, it can be seen that both (5R)-5-hydroxytriptolide (T8) and the reference substance (triptolide) can effectively inhibit the activity of the background NF-κB signaling pathway, and the activities of the two compounds are equivalent. The significant difference analysis in the inhibitory effects of the two compounds on the background NF-κB activity shows P=0.1, indicating no significant difference. (5R)-5-hydroxytriptolide (T8) and the reference substance (triptolide) can effectively inhibit the activity of the NF-κB signal pathway activated by TNF-α, and the activities of the two compounds are equivalent. The significant difference analysis in the inhibitory effects of the two compounds on the activity of NF-κB activated by TNF-α shows P=0.056, indicating no significant difference.

### Comparative example 3: the inhibitory effects of (5R)-5-hydroxytriptolide (T8) and the reference substance (triptolide) on immune cell activation

### 1. Experimental Materials

### 1) The Compounds to be Tested Were the Same as Above.

2) Cells and Reagents:

Peripheral blood mononuclear cells (PBMCs) were purchased from Shanghai Changhai Hospital, and the cells were cultured in RPMI-1640 complete medium containing 10% fetal bovine serum. 100 U/mL penicillin. 100 μg/mL streptomycin, and 20 IU/mL IL-2 (R&D).

RPMI-1640 medium and fetal bovine serum were purchased from Gibco: penicillin and streptomycin were purchased from Invitrogen: IL-2 was purchased from R&D: phorbol ester (Phorbol 12-Myristate 13-Acetate, PMA) and Ionomycin were purchased from Sigma: anti-CD3/CD28 antibody-coated microbeads and CD4 antibody-coated microbeads were purchased from MACS; flow cytometry antibodies CD3, CD4. CD38, and HLA-DR were purchased from eBioscience.

### 2. Experimental Method:

Freshly isolated healthy human PBMC (3×10⁷ cells) were cultured in cell culture medium containing IL-2 (20 IU/mL), stimulated with PMA (100 nM) and Ionomycin (1 μM) for 72 hours, and the compound to be tested (100 ng/ml) was added at the same time: the cells were washed twice with FACS buffer, followed by addition of anti CD3-PerCP, CD4-PE, CD38-PE-Cy7. HLA-DR-APC antibodies, stained for 30 minutes at 4° C.: the cells were washed twice with FACS buffer, and the expression of the above molecules was detected by FACS.

CD4⁺T-cells were isolated from freshly isolated healthy human PBMCs with anti-CD4 antibody-coated magnetic beads through positive screening and then cultured in a medium containing IL-2 (20 IU/mL). The cell activation was stimulated with anti-CD3/CD28 antibody-coated magnetic beads for 72 hours, and the compound to be tested was added at the same time. The cells were collected, washed twice with FACS buffer, followed by addition of anti CD3-PerCP, CD4-PE, CD38-PE-Cy7, HLA-DR-APC antibodies, stained at 4° C. for 30 minutes: the cells were washed twice with FACS buffer and collected, and the expression of the above molecules was detected by flow cytometry.

### 3. Experimental Results

By detecting the expression of cell activation indicators CD38 and HLA-DR, it is proved that (5R)-5-hydroxytriptolide (T8) and the reference substance (triptolide) can significantly inhibit the immune activation of CD4⁺T-cell subsets by PMA+Ionomycin or anti-CD3/CD28 antibody.

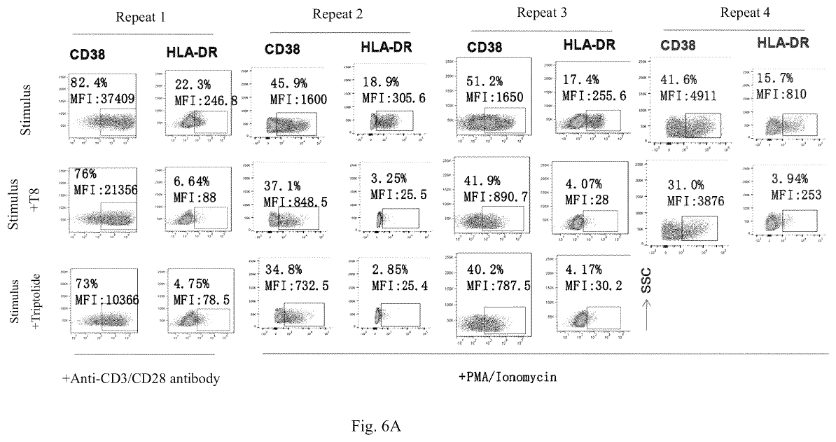

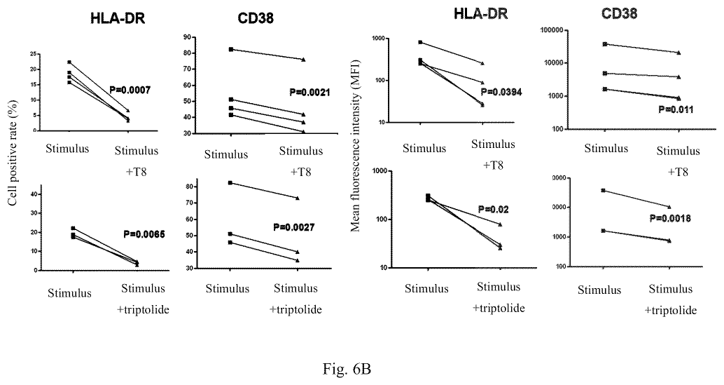

(5R)-5-Hydroxytriptolide (T8) and the reference substance (triptolide) have comparable effects in inhibiting the immune activation of CD4⁺T-cell subsets (see FIG. 6A and FIG. 6B for details).

### Comparative Example 4

1. Experimental materials:
1) The compounds to be tested is the same as above.
2) Cells and reagents:

Peripheral blood mononuclear cells (PBMCs) were purchased from Shanghai Changhai Hospital, and the cells were cultured in RPMI-1640 complete medium containing 10% fetal bovine serum, 100 U/mL penicillin, 100 μg/mL streptomycin, and 20 IU/mL IL-2 (R&D). The cells were cultured for 72 hours in a complete medium containing 5 μg/mL phytohemagglutinin (Phytohemagglutinin-P, PHA-P) to stimulated the proliferation of CD4⁺T-cells.

RPMI-1640 medium and fetal bovine serum were purchased from Gibco; Penicillin and streptomycin were purchased from Invitrogen: IL-2 was purchased from R&D; PHA-P was purchased from Sigma; CFSE dye was purchased from abcam: Anti-CD4-PE antibody was purchased from eBioscience.

### 2. Experimental Method:

Freshly isolated healthy human PBMC (1×10⁷ cells) were cultured in cell culture medium containing IL-2 (20 IU/mL), and were then stained with CFSE (10 nM) at 37° C. for 10 minutes. The cells were washed with the medium twice, PHA-P (5 μg/mL) was added, and the test compound T8 or triptolide (100 ng/mL each) was added at the same time. After 72 hours at 37° C. the cells were collected, stained with anti-CD4-PE antibody at 4°° C. for 30 minutes, and washed with FACS buffer twice. The cell proliferation was analyzed by flow cytometry.

### 3. Experimental Results

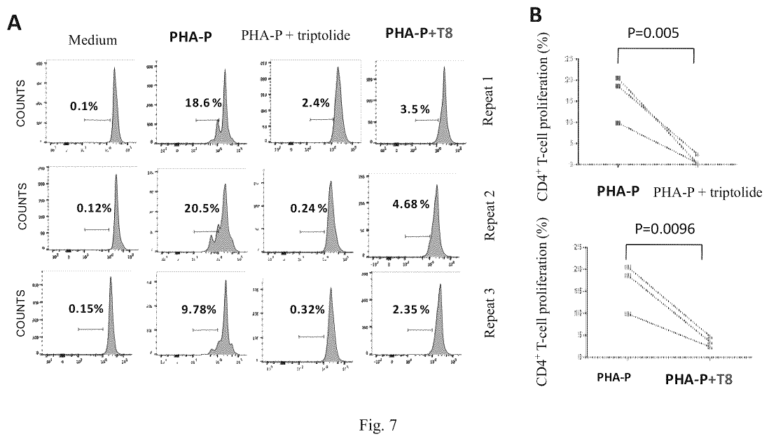

According to FIG. 7, it can be seen that (5R)-5-hydroxy triptolide (T8) and the reference substance (triptolide) can significantly inhibit the proliferation of CD4⁺T-cells in the healthy human peripheral blood and have a comparable effect.

Although the specific embodiments of the present disclosure are described above, those technician in this filed should understand that these are intended to be illustration, and various changes or modifications can be made to these embodiments without departing from the principle and essence of the present disclosure. Therefore, the protection scope of the present disclosure is defined by the appended claims.

### Comparative Example 5

The contents of the following reference are incorporated herein by reference in their entireties: Cao el. (5R)-5-hydroxytriptolide for HIV immunological nonresponders receiving ART: a randomized, double-blinded, placebo-controlled phase II study, the lancet, 2023, 34:1-12.

### 1. Experimental Materials:

The compounds to be tested is the same as above.

### 2. Participants

Eligible participants were people living with HIV (PLWH) aged 18-65 years old, who had received effective ART for at least four years and had plasma viral load below level of detection for at least 3.5 years, with their CD4 counts consistently below 350 cells/mm³. Patients were excluded if they had other types of immunodeficiency or severe co-morbidities. Those who received immunosuppressants or immunomodulators within six months before screening were also excluded. Contraception for participants during the study was required.

All participants provided written informed consent before participation in the study.

### 3. Experimental Method

The phase II, double-blind, randomized, placebo-controlled trial was conducted in adults patients with long-term suppressed HIV infection and suboptimal CD4 recovery, at nine hospitals in China.

The patients were 1:1:1 assigned to receive oral T8 0.5 mg or 1 mg daily, or placebo combined with antiretroviral therapy for 48 weeks. The study drug was recommended to be taken in the morning on an empty stomach. All study staff and participants were masked. The study drug was recommended to be taken in the morning on an empty stomach, and be separated from participants' daily ART. Participants were followed and assessed at Week 4, 12, 24, 36 and 48 of treatment. On each scheduled visit, the investigator would assess the participant's general status, newly-onset symptoms and signs, and adverse events.

On each visit, number of research drug pills taken since previous visit was assessed. In the final analysis, the exposure level of study drug was defined as the numbers of drug pills taken relative to the numbers prescribed. A level between 80 and 120% was considered acceptable for data interpretation, and the drug adherence is one of the PPS criteria.

Participants' ART mostly composed of two nucleoside reverse transcriptase inhibitors (NRTIs) combined with a non-nucleoside reverse transcriptase inhibitor (NNRTI), a boosted protease inhibitor (PI/r) or an integrase strand transfer inhibitor (INSTI), in multi-tablet or single-tablet formulation. Participants were encouraged to stay with original regimens, and modifications were allowed if needed for their HIV care.

### 4. Outcomes

The primary endpoints of the study included 1) Absolute change of CD4 counts from baseline to week 48: 2) Proportions of participants achieving a CD4 increase ≥50 cells/mm³ from baseline to week 48: and 3) changes of selected inflammatory markers from baseline to week 48, including interferon-y-induced protein 10 (IP-10), high-sensitivity C-reactive protein (hsCRP), and IL-6.

The secondary endpoints included 1) change in CD4/CD8 ratio from baseline to week 24 and week 48: 2) proportions of participants achieving a CD4 increase ≥20% from baseline to week 24 and week 48: 3) proportions of participants (baseline CD4 count <200 cells/mm³) achieving a CD4 count over 200 cells/mm³ at week 24 and 48, respectively.

Safety endpoints included occurrence of adverse events (AEs) and serious adverse events (SAEs) over the study period. Adverse events included those related to the study drug; all serious adverse events; and abnormal laboratory test values at each study visit.

### 5. Viro-Immunological Analysis

Plasma HIV-1 viral load was measured at Peking Union Medical College Hospital (PUMCH) Lab by COBAS AmpliPrep/COBAS TaqMan V2.0 RT-PCR (Roche). The lower detection limit is 20 copies per milliliter.

T-cell subsets were measured using CytoFlex flow cytometer (Beckman Coulter Life Science) at participating centers, including T cells (CD45+CD3+), CD4 T cells (CD45+CD3+CD4+), and CD8 T cells (CD45+CD3+CD8+). In PUMCH Lab. exploratory CD8 markers (CD45+CD3+CD8+CD38+ and CD45+CD3+CD8+HLA-DR+) were further measured for participants in three Beijing centers.

### 6. Satistical Analysis

The efficacy analysis was based on the full analysis set (FAS), which included all randomized patients with at least one posttreatment efficacy assessment, and the per-protocol set (PPS), which included patients who completed the study with proper study drug exposure and without any major protocol violations. The safety analysis was based on the safety analysis set (SAS), which included all randomized patients who received at least one dose of study drug. All safety results were descriptive, listed and summarized.

The primary efficacy differences between treatments on CD4 counts and inflammatory marker level were assessed through the t-test and analysis of covariance (ANCOVA) model. The ANCOVA model, including the baseline CD4 counts as the covariate, study group and center as fixed factors, generated the Least square means (LSMEANs) and relative 95% confidence interval (95% CI) in both FAS and PPS.

Treatment centers with less than 10 enrolled patients were merged in this model. Cochran-Mantel-Haenszel (CMH) chi-square test was used to compare the effects on CD4 improvement and other categorical data across different treatments in both FAS and PPS.

Missing values in primary efficacy analysis (i.e., CD4 counts and levels of inflammatory markers) were imputed with the last recorded data before the missing assessment. No imputation was performed in the secondary and exploratory analysis for missing values, differences between study groups were analyzed with unimputed data.

Statistical analysis was performed using SAS (version 9.4) and SPSS (version 25.0). All statistical tests were two-sided, and P values ≤0.05 were considered statistically significant for treatment differences.

### 7. Experimental Results

A total of 277 PLWH were screened at nine centers, and 151 eligible patients were recruited and randomized. Among them, two patients were found to have baseline CD4 counts over 350/mm³ before, so they did not receive assigned treatment and were excluded from the final analysis. A total of 46, 51, and 52 participants were allocated to LLDT-8 0.5 mg (LT8), LLDT-8 1.0 mg (HT8), and the placebo (PL) group. respectively.

Demographic and clinical features of all participants were summarized. The vast majority of participants were men (97.4%). The median age was 41 years old (range 22-64), with 61.1% under the age of 45. Participants received a median of 6.1 years of ART, ranging from 4.1 to 14.9 years. Most patients were receiving a NNRTI-based regimen (70.5%), most frequently the national free regimen tenofovir, lamivudine plus efavirenz (45.6%). Participants receiving boosted PI/r-based and INSTI-based regimens accounted for 18.1% and 11.4%, respectively. On enrollment, all participants were virologically suppressed, and the median CD4 count was 248 cells/mm³ (range 18-347), comparable among three study groups. Around ¼ participants had a CD4 count below 200 cells/mm³. Moreover, CD4 counts at two separate points before recruitment were also collected, showing a stable level during the previous year. The median baseline CD4/CD8 ratio was 0.45.

The analysis set for participants who underwent randomization in this study. Percentages may not total 100 because of rounding. Plus-minus values are means ±SD. BMI denotes body mass index; SD, standard deviation; ART, antiretroviral therapy; NNRTI, non-nucleoside reverse transcriptase inhibitor; TDF, tenofovir; 3TC, lamivudine; EFV, efavirenz; PI/r, ritonavir-boosted protease inhibitor; INSTI, integrase strand transfer inhibitor.

During the study, all participants remained virologically suppressed.

In the FAS population, the CD4 count at Week 48 was 303 cells/mm³ (95% CI 274, 333) in HT8 group, 299 cells/mm³ (95% CI 277, 320) in LT8 group, and 281 cells/mm³ (95% CI 261, 302) in PL group, respectively. The mean absolute increase of CD4 count from baseline was 63 cells/mm³ in HT8 group (95% CI: 41, 84), 49 cells/mm³ in LT8 group (95% CI: 29, 68), compared with 32 cells/mm³ in PL group (95% CI: 13, 51). All groups gained statistically significant CD4 increase compared to the baseline. However, CD4 increase in HT8 group was significantly higher than that of PL group (p=0.036). The LT8 group also gained more CD4 cells than PL group, but the difference was not significant at 48 weeks. The HT8 group exhibited a robust CD4 increase during the first month of treatment and kept relatively stable afterwards, while CD4 T cells in LT8 group tended to rise gradually over 48 weeks. In addition, changes of CD4 counts over the study were more consistent in the higher dose 1 mg group.

At Week 48, 23 of 46 (50%) participants in HT8, 23 of 51 (45.1%) in LT8, and 18 of 52 (34.6%) in PL group had a CD4 count increase over 50 cells/mm³. Both LLDT-8 groups had more participants achieving a discriminating CD4 increase, and the proportion increased with higher LLDT-8 dosing, but was statistically insignificant. At week 24, more participants in HT8 group achieved a CD4 increase >20%, however. such superiority became unremarkable at Week 48. Changes of CD4/CD8 ratio were comparable between the three groups at Week 48.

CD4 increase in the high-dose LLDT-8 group was more significant in participants over 45 years old. There were 17, 16, and 25 participants aged over 45 in the HT8, LT8 and PL groups, respectively. After 48 weeks of 1 mg LLDT-8 treatment, a mean increase of 96 cells/mm³ (95% CI: 54, 137) were achieved in participants over 45 years old, compared with 20 cells/mm³ in PL group (p<0.001).

The efficacy of LLDT-8 was also more prominent in participants with a baseline CD4 count 200-350 cells/mm³. In these participants, a consistent superiority of CD4 increase was observed in HT8 over PL. In contrast, in those with a baseline CD4 count below 200 cells/mm³, effect of LLDT-8 was insignificant at Week 48.

18 of 46 participants in HT8 group, 21 of 51 in LT8 group and 18 of 52 in PL group received inactive SARS-COV-2 vaccination (Sinopharm, or Sinovac) during the study. Among them, 10, 11 and 10 participants in HT8, LT8 and PL group received at least one vaccine within three months before the last visit. Stratified analysis showed that benefit from high-dose LLDT-8 in CD4 counts became insignificant in participants receiving SARS-COV-2 vaccination regardless of the time point.

At Week 48, the mean change of serum IP-10 level was −72.1 pg/ml (95% CI −97.7, −46.5) in HT8 group, −42.5 pg/ml (95% CI −66.8, −18.2) in LT8 group, and −22.8 pg/ml (95% CI −47.1, 1.5) in PL group, respectively. LLDT-8 treatment led to reduction of IP-10 levels in both treated groups, and 1 mg daily LLDT-8 induced more substantial IP-10 decrease compared with the placebo (p=0.007). HsCRP and IL-6 also showed slight decrease at Week 48 in the HT8 group, but with limited clinical indications since they were both within normal ranges.

Association between changes in IP-10 levels and CD4 counts at each time point were analyzed. Interestingly, dynamics of plasma IP-10 level changes showed similar shape to that of the CD4 count changes. Of note, in the HT8 and PL groups, levels of IP-10 decrease were significantly associated with CD4 increase (Pearson's r=0.18, p=0.005).

The cellular activation levels of CD8 T cells were measured in 62 participants from the three Beijing centers, including 19 in HT8 group, 21 in LT8 group and 22 in PL group. The surface expressions of CD38 or HLA-DR on CD8 T cells were comparable among the three groups.

All participants stayed virologically-suppressed during the study. Treatment-emergent adverse events (TEAEs) were reported in 41 of 46 (89.1%) participants in HT8 group, 43 of 51 (84.3%) in LT8, and 42 of 52 (80.7%) in PL group. A total of 610 events were reported, and 164 of them were presumably considered possibly, probably, or related to the study drug. The most frequently reported (>5%) drug-related TEAEs included hyperlipidemia, increased alanine transaminase (ALT), decreased white blood cells, decreased neutrophils, increased gamma-glutamyl transferase (GGT) and hepatic steatosis. Numbers of TEAEs were comparable between the three groups. TEAEs ≥Grade 3 were slightly more frequent in the HT8 group, mostly neutrophil decrease and dyslipidemia, yet no severe infection occurred, nor did any drug-related TEAEs lead to study discontinuation. No drug-related SAEs were reported.

A total of 149 patients were enrolled from and randomly allocated to receiving T8 0.5 mg daily (LT8, n=51), 1 mg daily (HT8, n=46), or placebo (PL, n=52). The median baseline CD4 count was 248 cells/mm³, comparable among three groups. T8 was well-tolerated in all participants. At 48 weeks, change of CD4 counts was 49 cells/mm³ in LT8 group (95% confidence interval [CI]: 30, 68), 63 cells/mm³ in HT8 group (95% CI: 41, 85), compared to 32 cells/mm³ in placebo group (95% CI: 13, 51). T8 1 mg daily significantly increased CD4 count compared to placebo (p=0.036), especially in participants over 45 years. The mean change of serum interferon-γ-induced protein 10 was −72.1 mg/L (95% CI-97.7,-46.5) in HT8 group at 48 weeks, markedly decreased compared to −22.8 mg/L (95% CI-47.1. 1.5, p=0.007) in placebo group. Treatment-emergent adverse events (TEAEs) were reported in 41 of 46 (89.1%) participants in HT8 group, 43 of 51 (84.3%) in LT8, and 42 of 52 (80.7%) in PL group. No drug-related SAEs were reported.

1.-16 (canceled)

17. A method for treating or preventing a disease of incomplete immune reconstitution in AIDS in a subject in need thereof, comprising administering a therapeutically effective amount of a medicament comprising (5R)-5-hydroxytriptolide to the subject.

18. The method according to claim 17, wherein the disease of incomplete immune reconstitution is associated with abnormal immune activation.

19. The method according to claim 18, wherein the abnormal immune activation is NF-κB-related abnormal immune activation.

20. The method according to claim 18, wherein the abnormal immune activation is chronic abnormal immune activation.

21. The method according to claim 17, wherein the subject is an AIDS patient who has received effective ART for at least 3 years and has plasma viral load below level of detection for at least 2 years, with their CD4 counts consistently below 350 cells/mm³, the CD4 counts are measured using CytoFlex flow cytometer in vitro before the administration.

22. The method according to claim 21, wherein the subject is an AIDS patient who has received effective ART for at least four years and has plasma viral load below level of detection for at least 3.5 years, with their CD4 counts consistently below 350 cells/mm³.

23. The method according to claim 17, wherein the method is to treat or prevent the disease of incomplete immune reconstitution in AIDS by enhancing CD4 counts, the CD4 counts are measured using CytoFlex flow cytometer in vitro before and after the administration.

24. The method according to claim 17, wherein the (5R)-5-hydroxytriptolide is in the form of solid tablet.

25. The method according to claim 17, wherein the (5R)-5-hydroxytriptolide is one of the active ingredients or the only active ingredient in the medicament.

26. The method according to claim 17, wherein the (5R)-5-hydroxytriptolide is administered orally.

27. The method according to claim 17, wherein the (5R)-5-hydroxytriptolide is administered as 0.5 mg-1 mg daily.

28. The method according to claim 17, wherein the (5R)-5-hydroxytriptolide is administered as 0.5 mg or 1 mg daily.

29. A method for enhancing CD4 counts in a subject in need thereof, comprising administering a therapeutically effective amount of a medicament comprising (5R)-5-hydroxytriptolide to the subject.

30. The method according to claim 29, wherein the subject in need thereof is the patient with a disease of incomplete immune reconstitution in AIDS.

31. The method according to claim 29, wherein the subject is an AIDS patient who has received effective ART for at least 3 years and has plasma viral load below level of detection for at least 2 years, with their CD4 counts consistently below 350 cells/mm³, the CD4 counts are measured using CytoFlex flow cytometer in vitro before the administration.

32. The method according to claim 31, wherein the subject is people living with HIV, who has received effective ART for at least four years and has plasma viral load below level of detection for at least 3.5 years, with their CD4 counts consistently below 350 cells/mm³.
